(19) United States
(12) Patent Application Publication
(10) Pub. No.: US 2007/0098703 A1
(43) Pub. Date: May 3, 2007

(54) Methods for Using Adipose-Derived Cells for Healing of Aortic Aneurysmal Tissue

(75) Inventors: Marc Hendriks, Brunssum (NL); Brian Kwitkin, Pembroke Pines, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/564,715

(22) Filed: Nov. 29, 2006

Related U.S. Application Data
(62) Division of application No. 10/422,176, filed on Apr. 23, 2003.

(51) Int. Cl. A61K 48/00; A61F 2/06

(52) U.S. Cl. 424/93.21; 128/898; 623/1.11; 623/1.41; 424/423

(57) ABSTRACT

The present invention encompasses methods and apparatus for minimizing the risks inherent in endovascular grafting for aneurysm repair. The invention includes tracking a delivery means into an aneurismal site and deploying a stent graft in the aneurysmal site along side the delivery means. Next, adipocytes derived from adipose tissue are delivered to the aneurysmal site.

### CROSS REFERENCE TO RELATED APPLICATIONS

This application is a divisional of U.S. patent application Ser. No. 10/422,176 filed Apr. 23, 2003, which is incorporated by reference herein in its entirety.

### BACKGROUND OF THE INVENTION

Aortic aneurysms represent a significant medical problem for the general population. Aneurysms within the aorta presently affect between two and seven percent of the general population and the rate of incidence appears to be increasing. This form of atherosclerotic vascular disease (hardening of the arteries) is characterized by degeneration of the arterial wall in which the wall weakens and balloons outward by thinning. Until the affected artery is removed or bypassed, a patient with an aortic aneurysm must live with the threat of aortic aneurysm rupture and death.

One known clinical approach for patients with an aortic aneurysm is a surgical repair procedure. This is an extensive operation involving dissection of the aorta and replacement of the aneurysm with an artificial artery known as a prosthetic graft. Such a procedure requires a significant incision to expose the aorta and the aneurysm so that the graft can be directly implanted. The operation requires general anesthesia with a breathing tube, drainage tubes, and extensive intensive care monitoring in the immediate post-operative period, along with possible blood transfusions. All of these procedures impose stress on the cardiovascular system.

Alternatively, there is a significantly less invasive clinical approach to aneurysm repair known as endovascular grafting. Endovascular grafting involves the transluminal placement of a prosthetic arterial graft in the endoluminal position (within the lumen of the artery). To prevent rupture of the aneurysm, a stent graft of tubular construction is introduced into the blood vessel, typically from a remote location through a catheter introduced into a major blood vessel in the leg. The catheter/stent graft is then pushed through the blood vessel to the aneurysm location, and the stent graft is secured in a location within the blood vessel such that the stent graft spans the aneurysmal sac. The outer surface of the stent graft, at its ends, is sealed to the interior wall of the blood vessel at a location where the blood vessel wall has not suffered a loss of strength or resiliency, such that blood flowing through the vessel is diverted through the hollow interior of the stent graft, and thus is diverted from the blood vessel wall at the aneurysmal sac location. In this way, the risk of rupture of the blood vessel wall at the aneurysmal location is significantly reduced—if not eliminated—and blood can continue to flow through to the downstream blood vessels without interruption. The stent graft is sized such that upon placement into an aneurysmal blood vessel, the diameter of the stent graft slightly exceeds the existing diameter of the blood vessel at healthy blood vessel wall site on opposed ends of the aneurysm.

An exciting area of tissue engineering is the emerging technology of “self-cell” therapy, where autologous cells of a given tissue type are removed from a patient, isolated and perhaps mitotically expanded or genetically engineered, and ultimately reintroduced into the donor/patient with or without synthetic materials or other carrier matrices. One goal of self-cell therapy is to help guide and direct the rapid and specific repair of tissues. Such self-cell therapy is already a part of clinical practice; for example, using autologous bone marrow transplants for various hematologic conditions. The rapid advancement of this technology is further reflected in recent publications that disclose rapid progress toward bone and cartilage self-cell therapy. Moreover, similar advances are being made with other tissues such as muscle, liver, pancreas, tendon and ligament. One of the greatest advantages of self-cell therapy over current technologies is that the autologous nature of the tissue/cells greatly reduces, if not eliminates, immunological rejection and the costs associated therewith.

One form of self-cell therapy that recently has received attention is based on the use of adipose tissue. Adipose tissue-based therapy and corresponding technologies have gained attention for a variety of reasons. First, adipose tissue is abundant in most human beings and the vast majority of humans have enough subcutaneous adipose tissue to donate the amount required for self-cell therapy without any significant biological or anatomical consequences. Second, adipose tissue is easily obtained through liposuction, a minimally invasive procedure. Moreover, when the liposuction procedure is combined with subcutaneous infiltration of anesthetic solution, it can be performed with the patient being awake or only minimally sedated.

Thus there is a desire in the art to achieve a greater success of aneurysm repair, using minimally invasive procedures and reducing or eliminating immunological rejection. The present invention satisfies this need in the art.

### SUMMARY OF THE INVENTION

The present invention addresses the problem of aneurysm repair, particularly the problem of endoleaks (blood leaking into the space between the outer surface of the stent graft and the inner wall of the aneurysmal sac) associated with the use of endovascular stent grafts for aneurysm repair. A consequence of such endoleaks, in addition to other complications of aneurysm repair, is rupture of the aneurysm. The present invention provides methods for supporting or bolstering the aneurysmal site with healthy tissue derived from self-cell therapy.

Thus, in one embodiment of the invention there is provided a method of repairing an aneurysm in an individual, comprising: harvesting adipose tissue from the individual; isolating adipocytes from the adipose tissue substantially free from other cell types; tracking a delivery means into an aneurismal site; deploying a stent graft in the aneurysmal site along side the delivery means; and delivering the isolated adipocytes to the aneurysmal site in the individual by the delivery means. In one embodiment of this aspect of the invention, the adipocytes are genetically engineered or expanded in vitro, and/or delivered in conjunction with a carrier and/or cellular scaffold. In yet another aspect of this embodiment of the invention, the delivery means is a catheter. Alternatively, adipogenic cells can be isolated, differentiated in vitro, then delivered to the aneurysmal site.

Another embodiment of the invention provides an apparatus for repairing an aneurysm, comprising: a stent graft; a delivery means; and adipocytes isolated from adipose tissue and substantially free of other cell types disposed within the delivery means.

### BRIEF DESCRIPTION OF THE DRAWINGS

A more particular description of the invention, briefly summarized above, may be had by reference to the embodiments of the invention described in the present specification and illustrated in the appended drawings. It is to be noted, however, that the specification and appended drawings illustrate only certain embodiments of this invention and are, therefore, not to be considered to be limiting of its scope. The invention may admit to equally effective embodiments as defined by the claims.

FIG. 1 is a schematic view of a human aortal aneurysm.

FIG. 2 is a partial sectional view of a descending aorta with a bifurcated stent graft placed therein.

FIG. 3 is a flow chart of one embodiment of the methods of the present invention.

FIG. 4 is a partial sectional view of a descending aorta with a bifurcated stent graft and a delivery catheter placed therein.

### DETAILED DESCRIPTION OF THE INVENTION

Reference will now be made in detail to exemplary embodiments of the invention. While the invention will be described in conjunction with these embodiments, it is to be understood that the described embodiments are not intended to limit the invention solely and specifically to only these embodiments. On the contrary, the invention is intended to cover alternatives, modifications, and equivalents that may be included within the spirit and scope of the invention as defined by the attached claims.

The present invention encompasses methods and apparatus for minimizing the risks inherent in endovascular grafting for aneurysm repair. The invention includes a method for tracking a delivery means (for example, a catheter) through the vascular system of an individual with the distal end of the catheter reaching into an aneurysmal sac, and implanting an endovascular stent in the aneurysmal sac in a normal manner along side the delivery means. Adipocytes derived from adipose tissue of the individual are delivered to the aneurysmal sac through the delivery means. The adipocytes may be derived directly from adipose tissue, or may be cultured, expanded or manipulated before delivery. In addition, the adipocytes may be delivered along with a natural or synthetic cellular scaffolding material and/or a delivery solution. In addition, adipogenic cells can be isolated and stimulated to differentiate into adipocytes in vitro before delivery to the aneurysmal site.

As stated previously, endovascular grafts have proven successful in patients with aortic aneurysms; however, in some cases prolonged endoleakage problems have been reported after endovascular graft implantation. Endoleakage is the leakage of blood into the lumen or space between the outer surface of the stent graft and the inner wall of the aneurysmal sac. Various attempts have been made to overcome endoleakage problems, but no method has been able to control this problem effectively. In the present invention, tissue engineering using self-cell adipose-derived adipocytes addresses this important problem.

Essentially, three major elements are considered in tissue engineering design: cells, extracellular matrices, and growth factors—and the compatibility thereof with each other and with the host. In some cases of vascular prosthesis graft implantation, the implanted graft is directly surrounded by connective tissues and/or organs on its outer surface, and these tissues or organs can supply the three factors to the implanted graft. In such cases, the outer surface of implanted prosthesis becomes covered with connective tissue within a certain period of time after implantation. However, grafts implanted in luminal surfaces are not directly surrounded by connective tissues or organs, are not contacted by cells, tissue or growth factors, and thus do not achieve good connective tissue formation on their outer surface. It is this same principle that explains why the inside luminal surface of a vascular prosthesis does not become covered with tissue after implantation.

When implanting grafts into an aortal aneurysm, the grafts that span the aneurysm are essentially in the lumen of the aneurysmal sac and generally are surrounded with fresh blood coagula adhering to the outer graft surface. In addition, there are old mural thrombi adhering to the inside luminal surface of the aneurysm. After endovascular graft insertion, the blood coagula and thrombi might be organized into connective tissue—depending on the size of the aneurismal sac—thereby shrinking the aneurysm. However, when endoleakage occurs, the blood coagula are always renewed and cannot be organized into tissue for any length of time. The methods and apparatus of the present invention provide a sort of “cellular filler” for the aneurysmal sac, thereby combating the effects of endoleakage.

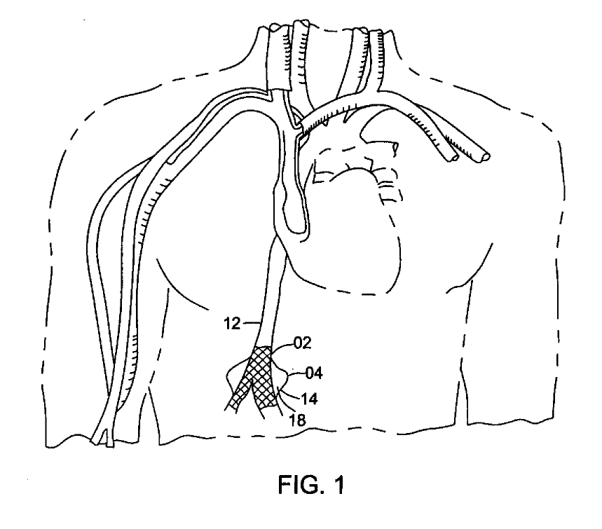

Referring initially to FIG. 1, there is shown generally an aneurysmal blood vessel 02; in particular, there is an aneurysm of the aorta 12, such that the aorta or blood vessel wall 04 is enlarged at an aneurysmal site 14 and the diameter of the aorta 12 at the aneurysmal site 14 is on the order of over 150% to 300% of the diameter of a healthy aorta 12. The aneurysmal site 14 forms an aneurysmal bulge or sac 18. If left untreated, the aneurysmal sac 18 may continue to deteriorate, weaken, increase in size, and eventually tear or burst.

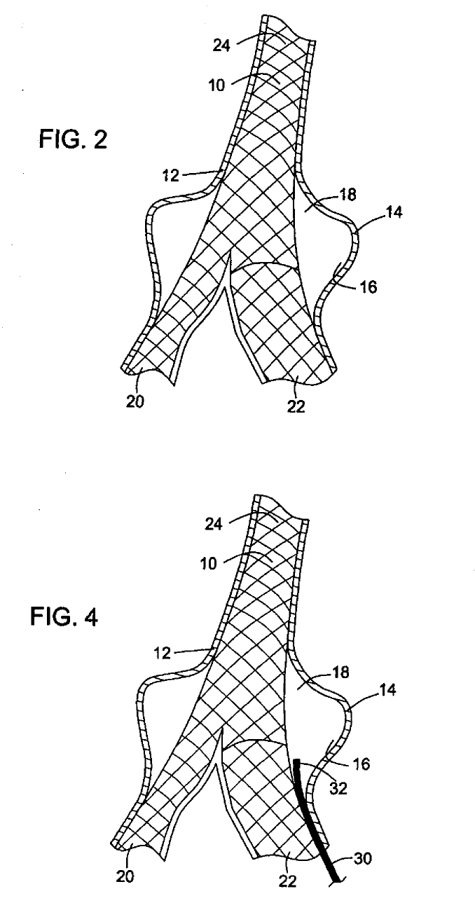

FIG. 2 shows the transluminal placement of a prosthetic arterial stent graft 10, positioned in a blood vessel, in this embodiment, in, e.g., an abdominal aorta 12. The prosthetic arterial stent spans, within the aorta 12, an aneurysmal portion 14 of the aorta 12. The aneurysmal portion 14 is formed due to a bulging of the aorta wall 16, in a location where the strength and resiliency or the aorta wall 16 is weakened. As a result, an aneurysmal sac 18 is formed of distended vessel wall tissue. The stent graft 10 is positioned spanning the sac 18 providing both a secure passageway for blood flow through the aorta 12 and sealing of the aneurysmal portion 14 of the aorta 12 from additional blood flow from the aorta 12.

The placement of the stent graft 10 in the aorta 12 is a technique well known to those skilled in the art, and essentially includes opening a blood vessel in the leg or other remote location and inserting the stent graft 10 contained inside a catheter (not shown) into the blood vessel. The catheter/stent graft combination is tracked through the remote vessel until the stent graft 10 is deployed in a position that spans the aneurysmal portion 14 of aorta 12. The bifurcated stent graft 10 shown in FIG. 2 has a pair of branched sections 20, 22, bifurcating from a trunk portion 24. This style of stent graft 10 is typically composed of two separate pieces, and is positioned in place first by inserting a catheter with the trunk portion 24 into place through an artery in one leg, providing a first branched section 20 to the aneurysmal location through the catheter and attaching it to the trunk portion at the aneurysmal site. Next, a second catheter with the second branched section 22 is inserted into place through an artery in the other leg of the patient, positioning the second branched section 22 adjacent to the trunk portion 24 and connecting it thereto. The procedure and attachment mechanisms for assembling the stent graft 10 in place in this configuration are well known in the art, and are disclosed in, e.g., Lombardi, et al., U.S. Pat. No. 6,203,568.

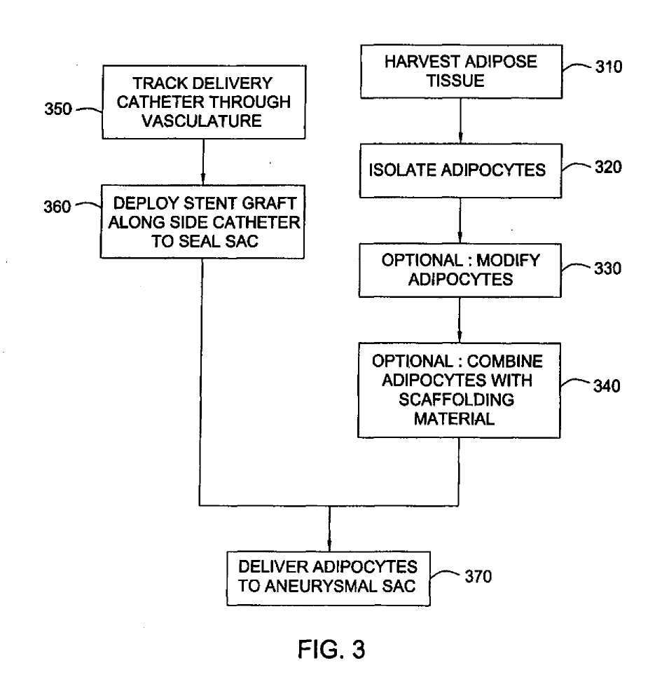

FIG. 3 is a flow chart of one embodiment of the methods of the present invention. In FIG. 3, method 300 is comprised of five main steps and two optional steps. In step 310, adipose tissue is harvested. Adipose tissue is readily accessible and abundant in most individuals and can be harvested by liposuction. Various liposuction techniques exist, including ultrasonic-assisted liposuction (“UAL”), laser-assisted liposuction, and traditional suction-assisted liposuction (“SAL”), where fat is removed with the assistance of a vacuum created by either a mechanical source or a syringe.

Each of the foregoing liposuction techniques may be used in conjunction with tumescent solution. Liposuction procedures that use a tumescent solution generally involve pre-operative infiltration of subcutaneous adipose tissue with large volumes of dilute anesthetic solutions. The tumescent solution usually is comprised of saline or Ringer's solution containing low doses of epinephrine and lidocaine (e.g., at a concentration of 0.025%-0.1% of the saline or Ringer's solution). The amount of tumescent solution infiltrated is variable, but typically is in ratios of 2-3 cc of infiltrate per 1 cc of aspirated adipose tissue. Some practitioners use tissue turgor as the endpoint for tumescent solution infiltration. The evolution of the tumescent technique has revolutionized liposuction by making it available on an outpatient basis. Specifically, it makes the use of general anesthesia optional in most cases thereby avoiding the associated risks and costs. (See, e.g., Rohrich, et al., Plastic and Reconstructive Surgery, 99:514-19 (1997)).

An advantage of using adipose tissue as a source of “cellular filler” is that, due to the abundance of adipocytes in adipose tissue, adipocyte harvest, isolation and genetic manipulation can be accomplished peri-operatively. Thus, it is not necessary for the patient to submit to the liposuction procedure on one day and the adipocyte implantation the next. The procedures can be performed sequentially within minutes or tens of minutes of one another.

In addition to being abundant and easy to procure, adipose tissue is a source of several different cell types, including adipocytes, and adipogenic cells, the precursors to adipocytes. Further, adipose tissue is a potential source of extracellular matrix components, bioactive growth factors, paracrine and endocrine hormones.

Thus, in a next step, step 320 of FIG. 3, adipocytes are isolated from the harvested adipose tissue. The harvested, isolated adipocytes preferably are cleaned and dissociated into smaller cell clumps, or even more preferably, single cell components. The dissociation step is accomplished by means known in the art such as by filtering, liquefying (enzymatic treatment of the harvested cells), or otherwise processing the harvested adipocytes. Adipocytes have been shown to exhibit increased cellular survival in vitro when such dissociation techniques are applied (see, e.g., Huss, F. R., and Kratz, G., Scand. J. Plast. Reconstr. Surg., 36(3):166-71 (2002)).

Adipocytes are identified by specific cell surface markers that react with unique monoclonal antibodies or with other compounds specific for the cell-surface markers. Homogeneous adipocyte compositions are obtained by the positive selection of adherent adipocytes that are free of cell-surface markers associated with other cell types present in the adipose tissue, such as hematopoietic cells, chondrocytes, osteocytes and other connective tissue-associated cells. For example, adipose differentiation-related protein (ADRP) is a 50 kDa membrane-associated protein whose expression is induced at the initiation of adipocyte differentiation and increases as pre-adipocytes continue to differentiate. Another protein that can be used to identify adipocytes is lipoprotein lipase, an early marker of adipocyte differentiation. Thus, adipocyte populations display epitopic characteristics associated only with adipocytes, and adipocyte isolation or purification can be accomplished by fluorescence activated cell sorting (FACS) or magnetic activated cell sorting (MACS) by techniques known by those skilled in the art. Alternatively, adipocyte isolation may be accomplished by growth in selective medium.

Step 330 of the present method shown in FIG. 3 allows for the option of modifying the adipocytes, such as genetically altering or engineering the adipocytes or expanding the adipocyte population in vitro. Methods for genetic engineering or modifying cells are known to those with skill in the art (see, generally, Maniatis, Fritsch & Sambrook, Molecular Cloning: A Laboratory Manual (1982); DNA Cloning: A Practical Approach, Volumes I and II (D. N. Glover, ed. 1985)); Embryonic Stem Cells, Methods and Protocols, (K. Turksen, ed., 2002). To genetically engineer the adipocytes, the adipocytes may be stably or transiently transfected or transduced with a nucleic acid of interest using a plasmid, viral or alternative vector strategy (see, e.g., Meunier-Durmont, C., et al., Eur. J. Biochem., 237(3):660-67 (1996) and Meunier-Durmont, C., et al., Gene Ther., 4(8):808-14 (1997)). Nucleic acids of interest include, but are not limited to those encoding gene products that produce or enhance the production of extracellular matrix components found in adipose tissue such as cytokines, growth factors, and angiogenic factors. For example, since tissue repair naturally occurs in an extracellular matrix environment rich in glycosamines and glycoproteins, it makes sense to genetically engineer the adipocytes to produce one or more such compounds.

In addition to growth factors, adipocytes may be engineered to express drugs or therapeutics useful in the aneurysm healing process such as tissue inhibitors of matrix metalloproteinases (TlIMPs) or other therapeutics.

The transduction of viral vectors carrying regulatory genes into the adipocytes can be performed with viral vectors (adenovirus, retrovirus, adeno-associated virus, or other viral vectors) that have been isolated and purified. In such techniques, adipocytes are exposed to the virus in serum-free media in the absence or presence of a cationic detergent for a period of time sufficient to accomplish the transduction.

The transfection of plasmid vectors carrying regulatory genes into the adipocytes can be introduced into the adipocytes by use of calcium phosphate DNA precipitation or cationic detergent methods or in three-dimensional cultures by incorporation of the plasmid DNA vectors directly into a biocompatible polymer. Preferably, for peri-operative cell transfection electroporation is used. Electroporation protocols are known in the art and can be found, e.g., in Maniatis, Fritsch & Sambrook, Molecular Cloning: A Laboratory Manual (1982); DNA Cloning: A Practical Approach, Volumes I and II (D. N. Glover, ed. 1985)); Embryonic Stem Cells, Methods and Protocols, (K. Turksen, ed., 2002). For the tracking and detection of functional proteins encoded by the introduced genes, the viral or plasmid DNA vectors can contain a readily detectable marker gene, such as green fluorescent protein or the beta-galactosidase enzyme, both of which can be tracked by histochemical means.

Another method for modifying the adipocytes prior to delivery to the aneuryismal sac is to expand the adipocyte population. Basically, the expansion process is accomplished by prolonged in vitro culturing of the adipocytes in the selective cell culture medium (i.e., the medium that stimulates adipocyte growth) from several to many successive cell passages. However, because adipose tissue yields a large number of adipocytes, in vitro expansion typically is not necessary to be effective in the methods of the present invention.

Alternatively, it may be desirable to isolate adipogenic cells and stimulate their differentiation in vitro prior to delivery to the aneurysmal sac. Lineage-specific differentiation of adipogenic cells can be induced via supplementation of the cell culture medium by various compounds. For example, differentiation of adipogenic cells is stimulated by supplementation with isobutyl-methyl xanthine (IBMX), dexamethasone or insulin. One with skill in the art can select the appropriate compounds to differentiate adipogenic cells into an adipocyte.

Step 340 of FIG. 3 is another optional step that provides combining the adipocytes to be transferred to the aneurysmal sac with a carrier or scaffolding compound. Many strategies in tissue engineering have focused on the use of biodegradable polymers as temporary scaffolds for cell transplantation or tissue induction. The success of a scaffold-based strategy is highly dependent on the properties of the material, requiring at a minimum that it be biocompatible, easy to sterilize, and, preferably, degradable over an appropriate time scale into products that can be metabolized or excreted. Mechanical properties are also of crucial importance in polymer scaffold design for the regeneration tissues such as connective tissue, adipose tissue or blood vessels. In addition, scaffold degradation rates should be optimized to match the rate of tissue regeneration. Moreover, ideally degradable scaffolding polymers should yield soluble, resorbable products that do not induce an adverse inflammatory response. For general information regarding tissue engineering, see Ochoa and Vacanti, Ann. N.Y. Acad. Sci., 979:10-26 (2002); Chaikof, et al., Ann. N.Y. Acad. Sci., 961:96-105 (2002); Griffith, Ann. N.Y. Acad. Sci., 961:83-95 (2002); Weiss, et al., U.S. Pat. No. 6,143,293; and Zdrahala, et al., U.S. Pat. No. 6,376,742.

In addition to porous scaffolds, the present invention contemplates using a gel scaffold. Such gels may be synthetic or semisynthetic gels that may not only stimulate cells through adhesion and growth factor moieties, but may also respond to cells by degrading in the presence of specific cell cues. In one particularly well-developed family of gels known in the art, the basic macromer unit is a linear or branched polyethylene oxide end-capped with chemically reactive groups. Such a gel is particularly flexible for use in the present invention as it is intrinsically non-adhesive for cells and the gel properties can be tailored: the consistency of the gel can be controlled by the size of the monomers and the gel thickness; controlled degradation may be had by including hydrolysable polyester segments or enzyme-cleavable peptides at the chain ends, and adhesion peptides can be included in the gel at a concentration to control cell interactions.

One type of gel particularly useful in the present invention is a stimuli-responsive polymer gel. Stimuli-responsive polymer gels are compounds that can be triggered to undergo a phase-transition, such as a solgel transition. This property aids in reducing the pressure required to get the polymer-cell suspension through the delivery catheter. A preferred system would be a polymer-scaffolding system that is liquid at room temperature and gels at a temperature slightly below body temperature. Alternatively, photopolymerizable gels may be employed.

Yet another type of gel particularly useful in the present invention is autologous platelet gel derived from the patient's own blood. Autologous platelet gel is a substance that is created by pheresing platelet-rich plasma from whole blood and combining it with thrombin and calcium to form a coagulum. Several studies have shown enhanced healing due to the presence of supraphysiological concentrations of a variety of growth factors. Polypeptide growth factors such as platelet derived growth factor, transforming growth factor α and β, epithelial growth factor, fibroblast growth factor, and others, serve as potent inducers of normal tissue repair. These growth factors are released by activated platelets, amongst others. Platelet derived growth factor in particular has been shown to enhance wound healing in several animal models and non healing wounds in humans.

As described previously, adipocytes respond to soluble bioactive molecules such as cytokines, growth factors, and angiogenic factors. Thus, for example, tissue-inductive factors can be incorporated into the biodegradable polymer of the scaffold, as an alternative to or in addition to engineering the adipocytes to produce such inductive factors. Alternatively, biodegradable microparticles or nanoparticles loaded with these molecules can be embedded into the scaffold substrate.

Referring again to FIG. 3, once adipocytes have been isolated and expanded, or adipogenic cells have been isolated and differentiated, they can be delivered to the aneurysmal site. To do so, first a delivery means, such as a catheter, is tracked through the vascular system of an individual by methods known in the art, so that the distal portion of the catheter resides in the aneurysmal portion of the aorta (350). In some embodiments, the catheter may be a double- or triple-lumen catheter, where one lumen may be used to contain a fiber optic means to view the aneurysmal sac. Once the distal end of the delivery means is residing the aneurysmal portion of the aorta, a stent graft is deployed spanning the aneurysm (360) (see also FIG. 2). As discussed previously, the deployment or placement of the stent graft in an aorta is a technique well known to those skilled in the art. Once the stent graft and delivery means are in position, the adipocytes can then be delivered to the aneurysmal sac (370). Alternatively, other methods known in the art may be used to deploy the adipocytes such as percutaneous laparoscopic delivery, using two or more catheters—one to deploy the stent graft and one to deliver the cells, microinjection, or other methods known to those with skill in the art.

As discussed, the adipocytes can be delivered with or without a cellular scaffolding or matrix element. In addition, the adipocytes likely will be delivered in a pharmaceutically acceptable solution or diluent. For example, the adipocytes may be delivered in a carrier of sterile water, normal saline or other pharmaceutically acceptable carrier, alone or in combination with a pharmaceutically acceptable auxiliary substance, such as a pH adjusting or buffering agent, tonicity adjusting agent, stabilizer, wetting agent, and the like.

FIG. 4 is similar to FIG. 2, showing the transluminal placement of a prosthetic arterial stent graft 10 positioned in an aorta 12. The stent spans, within the aorta 12, an aneurysmal portion 14 of the aorta 12. The aneurysmal portion 14 is formed due to a bulging of the aorta wall 16. As a result, an aneurysmal sac 18 is formed of distended vessel wall tissue. The stent graft 10 is positioned spanning the sac 18 providing both a passageway for blood flow through the aorta 12 and sealing of the aneurysmal portion 14 of the aorta 12 from additional blood flow from the aorta 12. In addition, FIG. 4 shows a portion of a catheter (30) along side of the stent graft 10. The catheter (30) has a distal end (32) that resides in the aneurysmal portion 14 of the aorta 12. The adipose-derived adipocytes or in vitro-differentiated adipocytes from adipogenic cells are delivered to the aneurysmal site through the distal end (32) of the catheter (30). The adipocytes delivered may or may not be bioengineered, and may or may not be accompanied by cellular scaffolding, delivery solutions and/or soluble bioactive molecules such as cytokines, growth factors, and angiogenic factors or drugs. The adipocytes and other elements, if present, support or bolster the aneurysm, while providing the factors necessary to stimulate the growth of new tissue to continue to support the aneurysm.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

All references cited herein are to aid in the understanding of the invention, and are incorporated in their entireties for all purposes.

What is claimed is:

1-41. (canceled)

42. A method of repairing an aneurysm in an individual comprising:
harvesting adipose tissue from said individual;
isolating adipocytes from said adipose tissue;
combining said adipocytes with a biodegradable scaffolding material,
tracking a delivery means into said aneurysm;
deploying a stent graft along side said delivery means; and
delivering said isolated adipocytes and said biodegradable scaffolding material to the aneurysm sac in said individual by said delivery means.

43. The method of claim 42, wherein said biodegradable scaffolding material is a gel.

44. The method of claim 43, wherein said gel is a photopolymerizable gel, a stimuli-responsive gel or autologous platelet gel.

45. The method of claim 44 wherein said gel is autologous platelet gel.

46. The method of claim 42, wherein the scaffolding material comprises at least one cellular factor selected from the group of cytokines, growth factors, matrix metalloproteinase inhibitors or angiogenic factors.

47. The method of claim 42, wherein the delivery means is a catheter.

48. The method of claim 47, wherein the catheter is a multi-lumen catheter.

49. The method of claim 42, wherein the delivered adipocytes and scaffolding material substantially the aneurysm.

50. The method of claim 42, wherein the harvesting step is accomplished by liposuction.

51. The method of claim 42, wherein the adipogenic cells are isolated by growth in selective medium, by fluorescence activated cell sorting or by magnetic activated cell sorting.

52. An apparatus for repairing an aneurysm, comprising:
a stent graft;
a delivery means;
a biodegradable scaffolding material; and
autologous adipocytes disposed within the delivery means.

53. The apparatus of claim 52, wherein the scaffolding compound is a gel.

54. The apparatus of claim 53, wherein the gel is a photopolymerizable gel, a stimuli-responsive gel or autologous platelet gel.

55. The apparatus of claim 54 wherein said gel is autologous platelet gel.

56. The apparatus of claim 52, wherein the scaffolding material further comprises at least one cellular factor selected from the group of cytokines, growth factors, matrix metalloproteinase inhibitors or angiogenic factors.

57. The apparatus of claim 52, wherein the adipocytes have been genetically engineered.

58. The apparatus of claim 57, wherein the adipocytes are genetically engineered to produce at least one cellular factor selected from the group of cytokines, growth factors, matrix metalloproteinase inhibitors or angiogenic factors.

59. An apparatus for repairing an aneurysm, comprising:
a stent graft;
a delivery means;
autologous platelet gel; and
autologous adipocytes disposed within the delivery means.
